(19) United States
(12) Patent Application Publication
(10) Pub. No.: US 2024/0148412 A1
(43) Pub. Date: May 9, 2024

(54) STABLE LOCKING FIXATION OF THREADED BONE PINS AND OTHER COMPONENTS OF RING EXTERNAL FIXATOR

(71) Applicant: Kiran Saldanha Limited, Liverpool (GB)

(72) Inventors: Kiran Antony Nelson SALDANHA, Liverpool (GB); Yogesh Rao Narayan KALE, Bengaluru (IN)

(21) Appl. No.: 18/283,734

(22) PCT Filed: Mar. 22, 2022
(86) PCT No.: PCT/IB2022/052599

§ 371 Date: Sep. 22, 2023

(30) Foreign Application Priority Data
Mar. 22, 2021 (IN) 202141012126

(51) Int. Cl. A61B 17/62; A61B 17/64

(52) U.S. Cl. CPC A61B 17/62; A61B 17/645; A61B 17/6458

(57) ABSTRACT

A locking mechanism provides rotational stability to the threaded bone pins and other fixator components by modifying the opposing surfaces of the pin fixation bolt, the ring and other fixator components at their interface so that they can interlock with each other. A pin fixation bolt can be used to connect different diameter threaded bone pins. A multipurpose post enables variable angle and rotationally stable insertion of threaded bone pin and versatile use of the multipurpose post to replace currently available various fixator components. Plates enable rotationally stable, variable angle and variable position connection of the plates so that rings offset to each other can be connected without using oblique supports. Reversibly lockable hinges allow the hinge articulation to be locked or unlocked as desired, during the course of the treatment.

### PRIORITY

This application claims the priority of Indian patent application 202141012126, filed Mar. 22, 2021, which is hereby incorporated herein by reference in its entirety.

### FIELD

The present invention relates to ring external fixators and reversible locking mechanisms of the same.

### BACKGROUND

Ring external fixators, also called circular external fixators, provide multiplanar fixation of bone. They are used to stabilise fractures and to correct bone deformity. The treatment can sometimes last several months in the fixator while the patients are encouraged to weight bear to stimulate bone healing. As per the original method of ring external fixation proposed by Ilizarov, fine wires are passed from one side of the limb to the other side through the bone and the two ends of the wires are connected to the rings placed around the limb. This is called trans-osseous fixation. The attachment of fine wires to the ring is made using wire fixation blots that are inserted into the holes in the ring and secured with nuts. Attaching the fine wire to two points in the ring enables rotational stability of the wire. But trans-osseous wires are not well tolerated by patients especially in the thigh and upper arm. The safe corridors available for passing fine wires through the limb from one side to the other avoiding injury to critical structures such as nerves and blood vessels are narrow, as the surgeon must consider the critical structures on both sides of the limb.

Threaded bone pins, also called transcutaneous bone screws or half pins, are sometimes used instead of fine wires. The threaded bone pins inserted into the bone from one side of the limb, do not exit on the other side. The advantage of threaded bone pins is that the surgeon needs to consider critical structures only on one side of the limb, increasing the available safe corridors for insertion. Threaded bone pins are also more tolerated by patients especially in certain regions such as thigh and upper arm. Threaded bone pin is attached to the ring at a single point on the ring using a pin fixation bolt inserted into one of the holes in the ring and secured by tightening a nut around it. The disadvantage of this method of fixation is that the pin fixation bolt can rotate around its axis as there is no locking mechanism at the interface between the ring and the pin fixation bolt to resist rotational forces. The stability of the interface relies on friction between the smooth surface of the ring and the smooth surface of the pin fixation bolt, created by tightening the nut. When the ring fixator is subjected to repetitive loading over a prolonged period, the pin fixation bolt can become loose due to the rotational forces on it. Pin loosening increases the incidence of pin site infections and destabilises the external fixator construct resulting in a mechanically unstable environment that is detrimental to bone healing.

Several other components of ring external fixator such as posts, plates, hinges, sockets, cubes, and oblique supports are also connected to the ring and to each other simply by tightening them against each other. The surfaces at the interface between the ring and the fixator components and, at the interface between any two components are smooth and devoid of any form of locking mechanism to resist rotational forces. As a result, most of these components of the ring fixator are prone to loosening over a period.

In some recent modifications, the cubes are designed as double column cubes so that they can be attached to two holes or slots in the ring to provide rotational stability. This design makes it necessary to have a wider ring with two rows of holes or slots in the ring and makes the cubes heavier increasing the overall weight and bulk of the fixator. The ability of the double cubes to be oriented to different directions is limited by the two rows of holes and slots in the ring. The threaded bone pin can only be attached to the double column cube in its channel at a fixed angle.

Currently available components of ring external fixators such as posts, plates, sockets, cubes, and oblique supports enable surgeons to construct fixators with different configurations. For this purpose, different sizes (lengths) of these components must be available during surgery, increasing the total number of components that need to be held in stock and sterilised for each surgery. Similarly, different sizes of pin fixation bolts must be available to secure different diameter threaded bone pins.

Hinges are used in ring external fixators to allow movement at the hinge articulation during gradual correction of bone deformity and to enable joint movements when the fixator is applied across the joints. Sometimes after correction of bone deformity, while waiting for the bone to heal during the consolidation phase, the movement at the hinge articulation needs to be stopped. Currently this is performed by tightening the bolt and nut at the hinge articulation. The interface at the articulation of hinge joint does not have a true locking mechanism. Instead, the locking of hinge relies on the friction created between the two smooth surfaces of the opposing hinges at the articulation interface. As a result, the bolt and nut at the hinge articulation can become loose over a period causing unwanted movement of the hinge joint.

U.S. Pat. No. 5,681,309 teaches a distraction apparatus for an external fixation device used to rigidly immobilize bone fragments during bone lengthening and angular deformity correction includes distractor mechanisms and pivotable spacers for connecting frame members of an external fixator, and wire and half pin fasteners. The interconnected distractor mechanisms, pivotable spacers and frame members form a rigid structure with rigidity which is maintained during adjustment of the distraction.

The aforementioned invention mainly refers to a distractor mechanism that is unrelated to present invention. However, there is also a description of a pin fixation bolt with tear drop shaped hole to accommodate pins with different diameters. Tear drop hole provides three-point contact of surfaces to secure the pin whereas in the present invention the pin fixation bolt enables larger area of contact between the pin and the bolt by incorporating adapter half sleeves matching the diameter of different pins. In addition, the design feature in the present invention enables rotationally stable locking fixation of pin fixation bolt.

U.S. Pat. No. 5,630,814 discloses a fastener for wires or pins used with an external fixation device to rigidly immobilize bone fragments during linear or angular distraction thereof includes a fixation bolt with either a transverse asymmetrical shank bore, or a transversely slotted bolthead for providing multiple contact surfaces to secure the wires or pins, regardless of their diameters. Alternatively, the fastener can include a washer or a threaded washer-like member having nonparallel opposing planar surfaces with a transverse slot in one.

The aforementioned invention refers to the design of a pin fixation bolt with a tear drop shaped hole that enables to accommodate pins with different diameters. Tear drop hole provides three-point contact of surfaces to secure the pin whereas the design of pin fixation bolt in the present invention enables larger area of contact between the pin and the bolt by incorporating adapter half sleeves matching the diameter of different pins. In addition, the design feature in the present invention enables rotationally stable locking fixation of pin fixation bolt.

U.S. Pat. No. 8,608,740 B2 discloses a bolt adapted to clamp a pin to an external fixator system. The bolt includes a shaft having a threaded section, a head section and a pin opening that is arranged opposite the threaded section and accommodates and clamps a pin or a wire to a plate or ring of an external fixator. The plate comprises at least one opening which accommodates the shaft and a surface against which the pin or the wire is clamped by the bolt. The pin opening comprises at least two edges extending essentially along the same direction as the pin opening Such that the pin or wire to be clamped is contact with said edges.

The aforementioned invention relates to a pin fixation bolt to clamp pins with different diameters by incorporating at least two edges within the hole of the bolt. Although it is possible to use pins of different diameters, the contact area between the pin fixation bolt and pin is limited to the edges in the hole whereas the design of pin fixation bolt in the present invention enables bigger surface area of contact between the pin and pin fixation bolt by incorporating adapter half sleeves matching the diameter of different pins.

U.S. Pat. No. 10,285,734 B2 discloses external fixation frames for correcting bone deformities. The external fixation frames include a top fixation ring and a bottom fixation ring coupled to one another by at least two struts. A half ring is coupled to bottom fixation ring. The bottom fixation ring is u-shaped. One or more rocker members may be coupled to the bottom fixation ring. The half ring may be hingedly coupled to the bottom fixation ring such that it can rotate with respect to the bottom fixation ring from a first position to a second position.

The aforementioned invention relates to hinged connection of rings. Although the hinges can be tightened to frictionally engage such that rotation of the first hinge portion in relation to second hinge portion is resisted, there is no true locking mechanism at the interface between the two hinge portions. Additional resistance against rotation may be provided by textured surfaces, such as ridges, but these do not provide precise interlocking between the opposing surfaces at the interface to completely stop movements of the articulation whereas the hinges in the present invention can be reversibly locked to stop all movements using the locking mechanism where the two opposing surfaces at the interface are modified to precisely interlock with each other or kept open to allow movement as desired. The hinges in this invention do not have a rotationally stable connection to the ring or other components whereas the present invention enables rotationally stable locking fixation of hinges and all other components.

U.S. Pat. No. 10,405,888 B2 discloses systems and methods for manipulating the orientation of a plurality of bone fragments with respect to one another. A bone transport frame including first and second rings, a plurality of elongate struts, and a plurality of ring transport assemblies for orienting a first bone segment with respect to a second bone segment is disclosed. A third ring may be included in the bone transport frame for orienting a third bone segment with respect to the first and second bone segments. Manipulation of an adjustable member of the bone transport frame can transport a ring in either a proximal or distal direction with respect to other rings of the frame. Manipulation of another adjustable member of the bone transport frame can translate a central axis of one of the rings either toward or away from the central axes of the plurality of elongate struts.

The aforementioned invention relates to manipulation of bone segments by manipulating ring and other fixator components using bone transport assemblies and strut assemblies. There is no locking mechanism between the ring and the pin fixation bolt and other components to make them rotationally stable whereas the present invention enables rotationally stable locking fixation of pin fixation bolt and other components.

U.S. Pat. No. 10,624,674 B2 introduces a device which includes a plurality of rings. Each ring has a first face, a second face, and at least one slot defined by first and second interior edges of the ring on opposing sides of the slot. The at least one slot penetrates from the first face to the second face. The first face of each ring has a first recess adjacent the slot on the first edge and a second recess adjacent the slot on the second edge. A plurality of posts join each one of the plurality of rings to an adjacent one of the plurality of rings.

The aforementioned invention relates to a modified design of rings and other components. The rings have slots that allow variable positioning of components but there is no provision for variable angle locking of the components to the ring. Pin fixation bolt and all other components including posts, cubes do not have a locking mechanism to provide rotational stability. The present invention has locking mechanism for rotational stability between the ring and all components.

US Patent Application Pub. No: US 2020/0069349 A1 teaches a half pin for use in conjunction with external fixators, in which the half pin has a thin hydroxy apatite coating over a substantial portion thereof, a not—sharp hole—finding tip, and a variable pitch pattern in the threaded portion of the half pin. The hydroxyapatite coating must be thin—40-70 microns, deposited by plasma spray—and with limited porosity and pore size, and must extend not only over all the threads of the half pin but also a substantial portion of the unthreaded shaft proximal to the threaded end. The invention also includes a pin cube for use in association with the coated half pins Wires may also be coated with the hydroxyapatite coating.

The aforementioned invention relates mainly to a threaded bone pin designed to improve fixation at the bone pin interface. There is also a double column cube design proposed in this invention which will provide rotational stability to the pin. The disadvantage of the double column cube is that it requires a wider ring with two rows of holes or slots for attachment. There is limited arc within which variable angle positioning of double column cube is possible when attached to the slot in the ring, but it is not possible to attach it in any desired angle outside this arc. Variable angle attachment of the pin to double column cube is not possible, i.e., the angle between the pin and the double column cube remains fixed.

### SUMMARY

The present invention is designed to incorporate a locking mechanism to provide rotational stability to the threaded bone pins by modifying the surfaces of the pin fixation bolt and ring at their interface so that they can interlock with each other. Rotational stability of threaded bone pins reduces the chances of pin loosening and pin site infection. Similar locking mechanism at the interfacing surfaces of fixator components such as posts, plates, and hinges provide rotational stability to these components when they are connected to the ring or to each other making the whole construct more stable with a favourable biological environment for bone healing.

The locking mechanism in the present invention is also designed to enable orientation of threaded bone pins and other fixator components to any desired direction before locking. This feature enables variable angle insertion of threaded bone pins. It also improves the versatility of components so that surgeons can build different fixator configurations with fewer types of components. For example, the multi-purpose post of the present invention can be used not only as a post but also as a socket or as a cube, reducing the need for keeping large number of various components in stock and sterilising them for every surgery. In addition, pin fixation bolt in the present invention is designed to be used for threaded bone pins of different diameters eliminating the need for keeping pin fixation bolts of different sizes in stock. The locking mechanism of the plates in the present invention enables rotationally stable construction of fixator with rings that are offset to each other, without using oblique supports. The plate is designed to enable variable angle and variable position locked connection between it and the ring or other components.

The locking mechanism of the hinges in the present invention is designed to reversibly lock and unlock the hinge articulation as desired. This enables the surgeons to keep the hinges unlocked during the deformity correction phase and then lock them during the bone consolidation phase. Similarly, the hinges applied across a joint can be initially kept locked to immobilise the joints while the ligaments are healing and unlock them later to enable joint movement.

The present invention in certain embodiments overcomes the limitations of the prior art by:
Introduction of locking mechanism at the interface between the ring and the pin fixation bolt, by modifying their opposing surfaces at the interface, so that the two opposing surfaces interlock with each other to provide rotational stability for the threaded bone pin
Introduction of locking mechanism at the interface between the ring and other components including multipurpose posts, plates and hinges by modifying their opposing surfaces at the interface, so that the opposing surfaces interlock with each other to provide rotational stability for the components
Introduction of locking mechanism at the interface between all components including multipurpose posts, plates, hinges by modifying the opposing surfaces at the interface between the components, so that when any two of the components are connected to each other, their opposing surfaces interlock with each other to provide rotational stability
Design of a multipurpose post which can be used to perform the function of multiple prior art components:
   It can be used as a post to connect other components offset from the ring
   It can be used as a socket to connect rings or threaded rods
   It can be used as a cube for fixation of threaded bone pins offset from the ring.
   The multipurpose post has additional advantages over conventional cube:
      Locking mechanism at the interface between multipurpose post and ring provides rotational stability whereas a conventional cube does not have locking mechanism to prevent rotation
      Locking mechanism at the interface between the multipurpose post and pin fixation bolt enables insertion of threaded bone pins at any angle (variable angle) whereas conventional cubes enable insertion of threaded bone pins only at a fixed angle i.e. perpendicular to the cube
Design of a pin fixation bolt that can be used to connect threaded bone pins of different diameters using appropriate diameter adapter half sleeves
Design of a plate with a variable position locking slot and locking round holes that enable variable position locked connection of components
Introduction of a reversible locking mechanism to the articulation of the lockable hinge which enables the hinge articulation to be kept unlocked to allow movement during gradual correction of bone deformity. After completion of correction of bone deformity, the hinge articulation can be locked to stop movement during the bone consolidation phase. The opposing surfaces of the lockable hinge at the articulating interface are modified so that they can interlock with each other to stop all movements of the hinge articulation. Lockable double hinge has two articulations with their axes perpendicular to each other. The two articulations of lockable double hinge have separate, independent, reversible locking mechanisms.

Certain embodiments of the invention enable rotational stability at the interface between the ring, pin fixation bolt and various other components of the ring external fixator which results in:
reduced incidence of threaded bone pin loosening
reduced incidence of pin site infection
maintenance of overall stability of fixator construct
better healing of bone in a stable environment
reduced complications such as non-union, delayed union, malunion and deep infection

Another object of the invention is to design a pin fixation bolt that can be used to connect different diameter threaded bone pins.

Another object of the invention is to design a multipurpose post that enables:
variable angle and rotationally stable insertion of threaded bone pin
versatile use of multipurpose post to replace currently available various conventional fixator components such as posts, sockets and cubes

Another object of the invention is to design plates that enable rotationally stable, variable angle and variable position connection of the plates, so that rings that are offset to each other can be connected without using oblique supports.

Another object of the invention is to design reversibly lockable hinges that allow the hinge articulation to be locked or unlocked as desired, during the course of the treatment.

### BRIEF DESCRIPTION OF THE DRAWINGS

FIG. 1 shows a construct of rings connected with threaded rods and different types of rings.

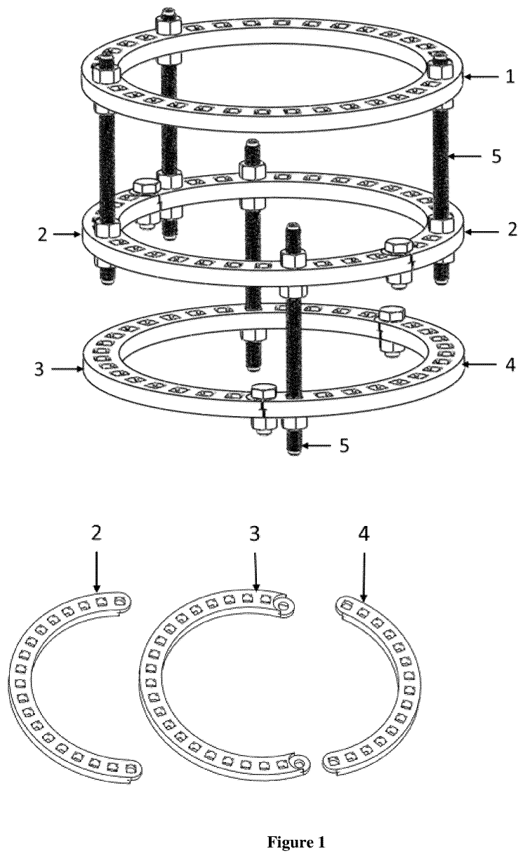

FIG. 2 shows different parts of the ring (inset shows enlarged view of part of the ring).

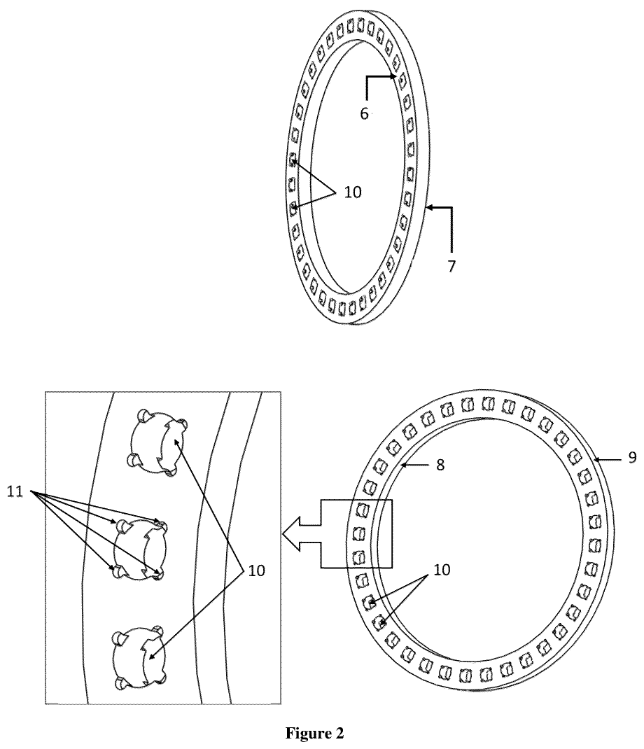

FIG. 3 shows two faces of the locking washer.

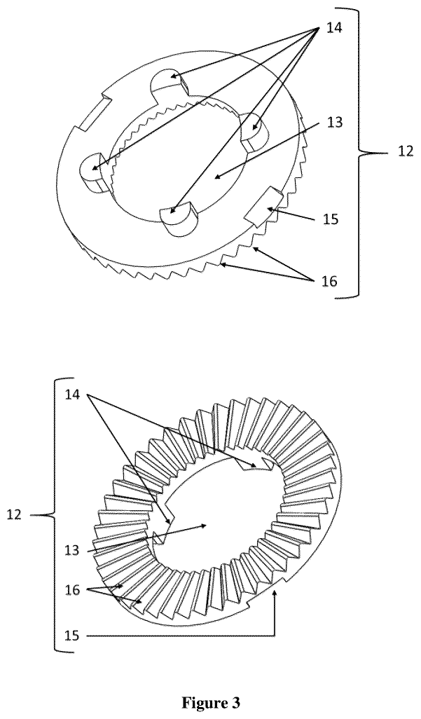

FIG. 4 shows locking washer mounted on a ring (inset shows enlarged view).

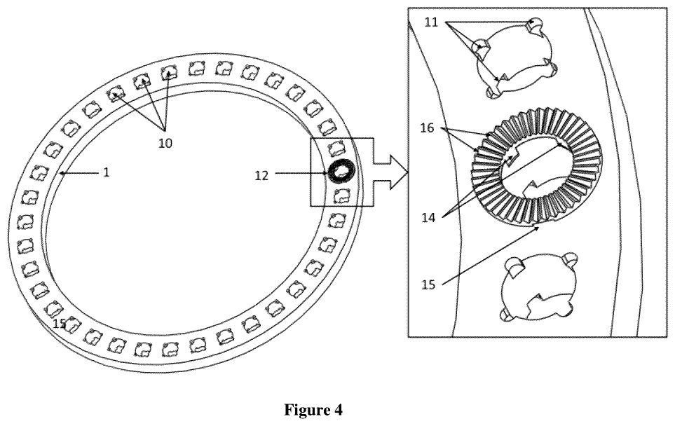

FIG. 5 shows an assembled view and exploded view of the pin fixation bolt.

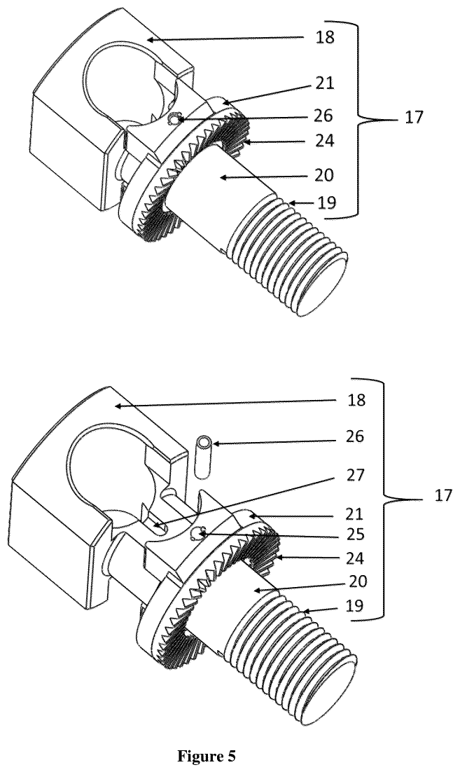

FIG. 6 shows different parts of pin fixation bolt.

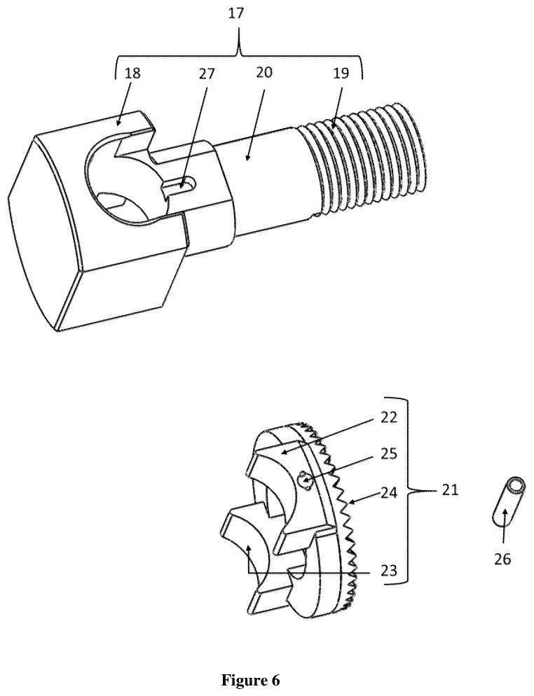

FIG. 7 shows the pin fixation bolt with a threaded bone pin mounted on a ring (inset shows exploded view).

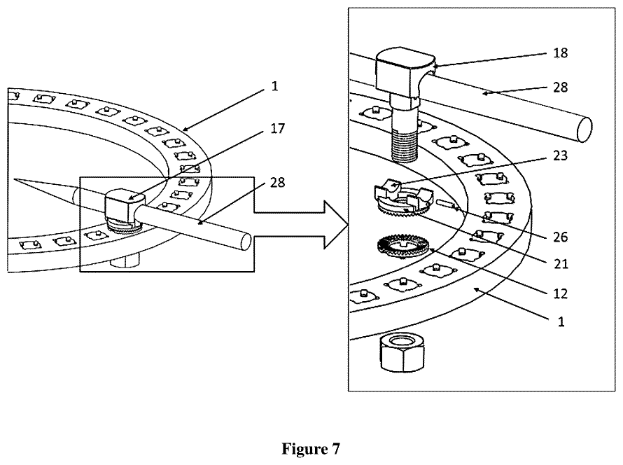

FIG. 8 shows an adapter half sleeve for smaller diameter threaded bone pin.

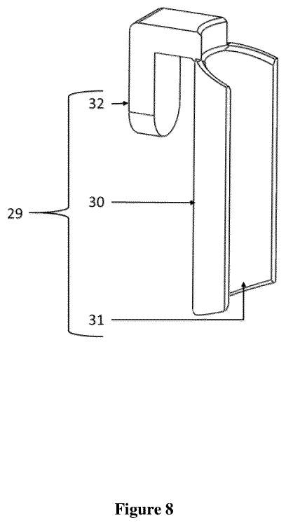

FIG. 9 shows an adapter half sleeve holding a small diameter threaded bone pin in a pin fixation bolt (inset shows exploded view).

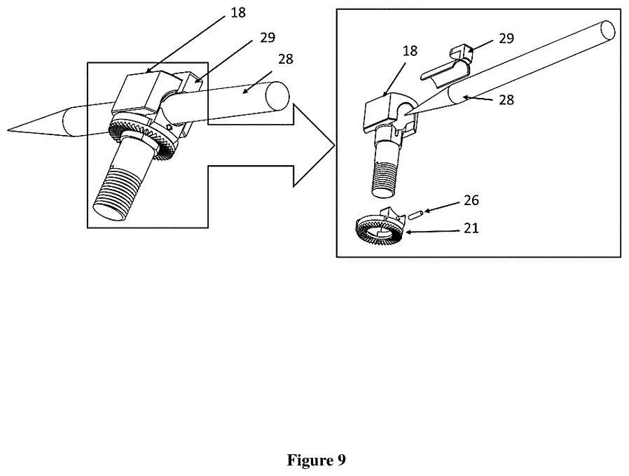

FIG. 10 shows a top view and a bottom view of multipurpose post.

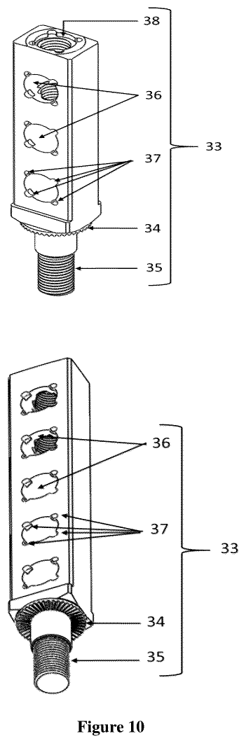

FIG. 11 shows a multipurpose post mounted on a ring with a pin fixation bolt attached to one of the holes in its body and a threaded rod attached to its top.

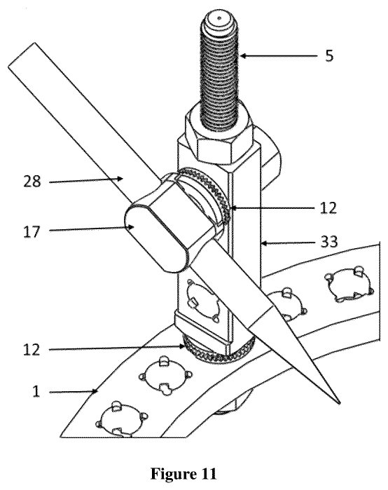

FIG. 12 shows two multipurpose posts connecting two rings (inset shows enlarged view of the connection).

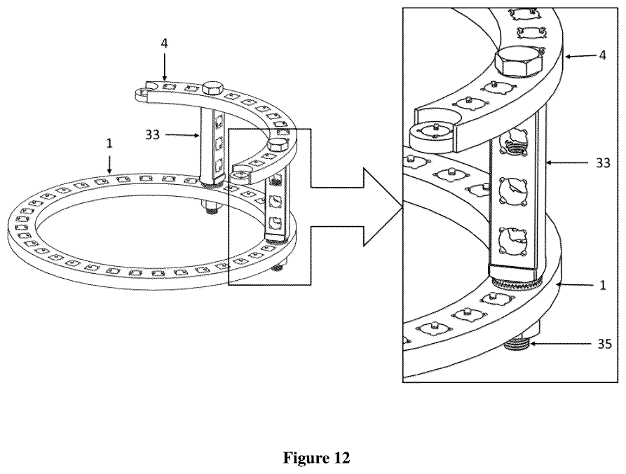

FIG. 13 shows different parts of a plate.

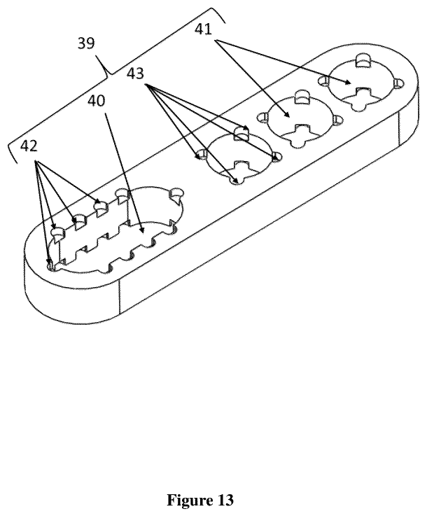

FIG. 14 shows locking washers mounted at different positions on a plate.

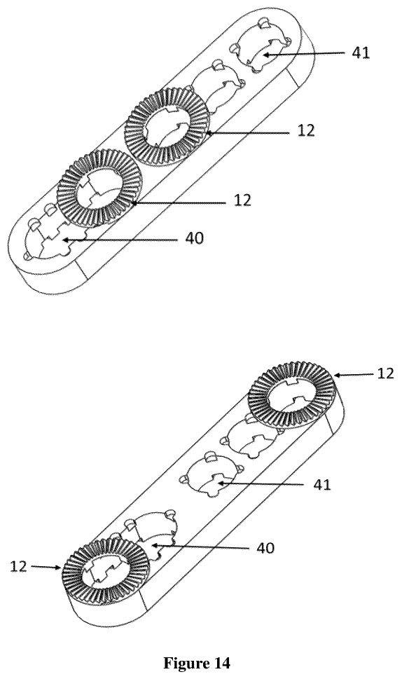

FIG. 15 shows two plates connecting two rings offset to each other (inset shows exploded view of the connection).

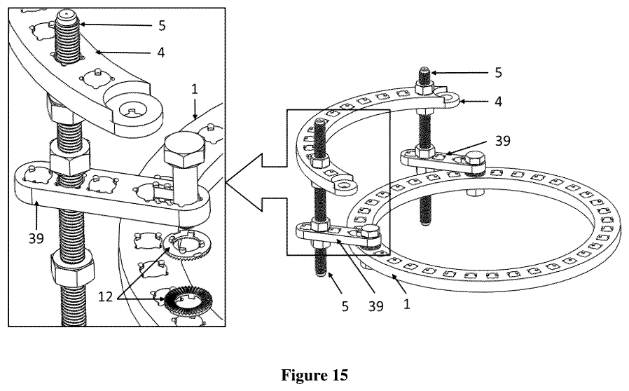

FIG. 16 shows the plate mounted on a multipurpose post (inset shows exploded view of the connection).

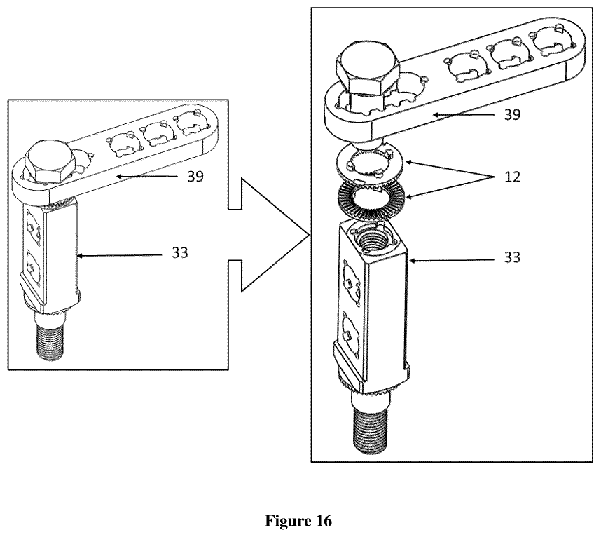

FIG. 17 shows two faces of a lockable hinge assembly.

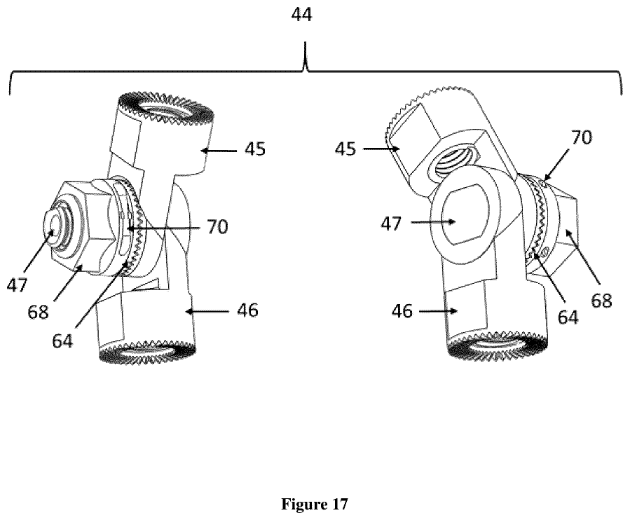

FIG. 18 shows two faces of first articulating part of the lockable hinge.

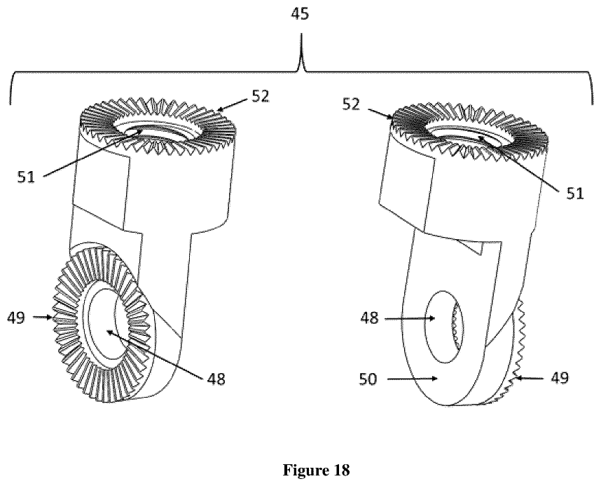

FIG. 19 shows two faces of second articulating part of lockable hinge.

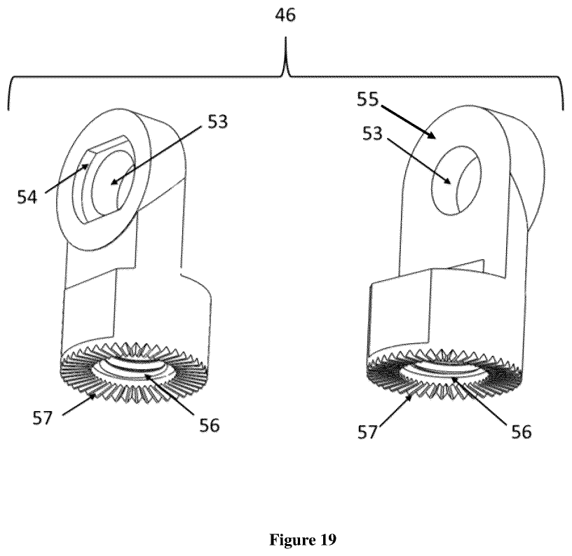

FIG. 20 shows a central link shaft of lockable hinge.

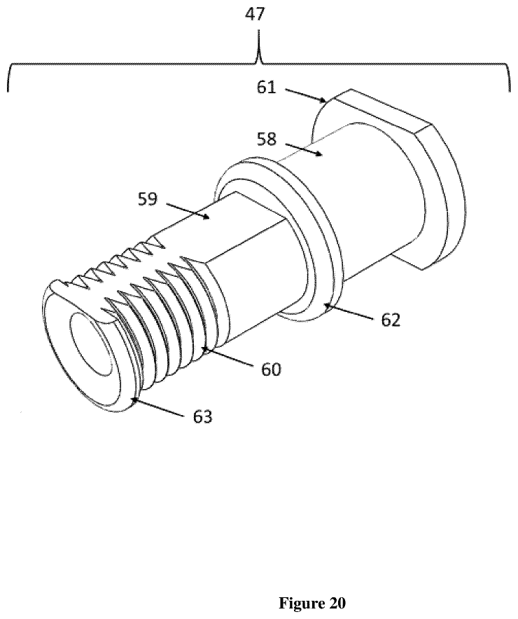

FIG. 21 shows two faces of sliding washer of lockable hinge.

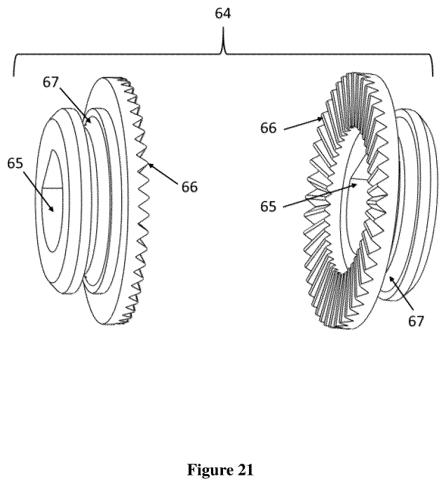

FIG. 22 shows two faces of an activator nut and, retainer C-pin of lockable hinge.

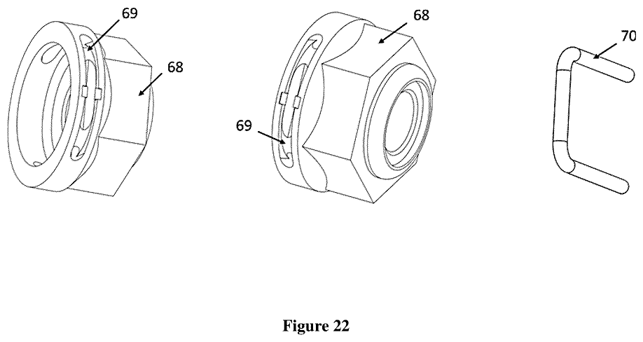

FIG. 23 shows an exploded view of lockable hinge.

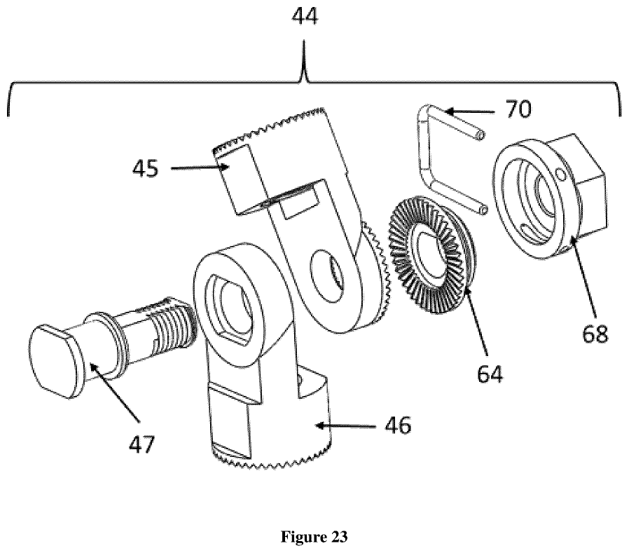

FIG. 24 shows a lockable double hinge assembly.

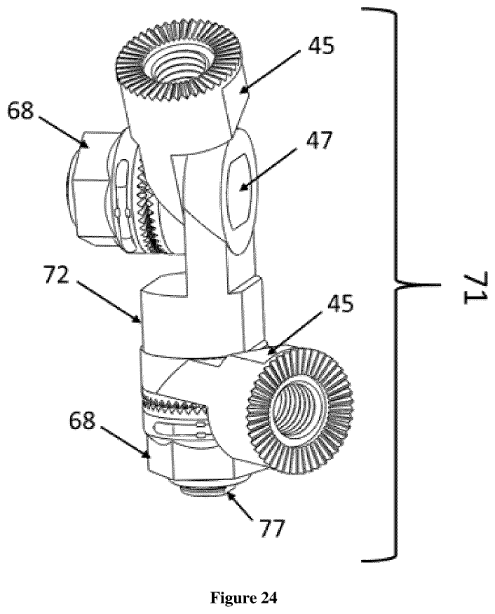

FIG. 25 shows an exploded view of lockable double hinge.

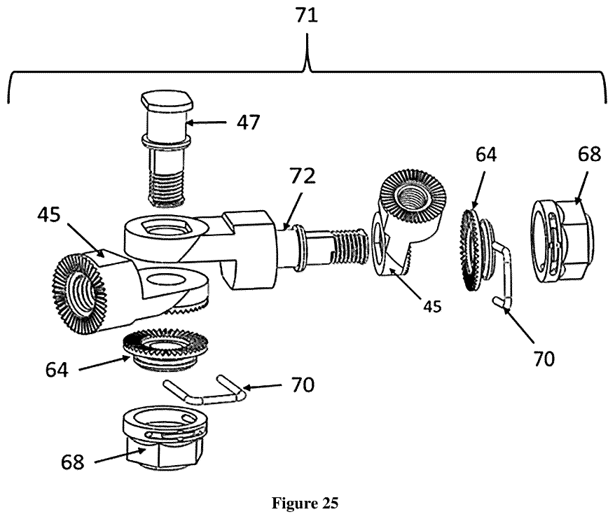

FIG. 26 shows two faces of middle articulating part of lockable double hinge.

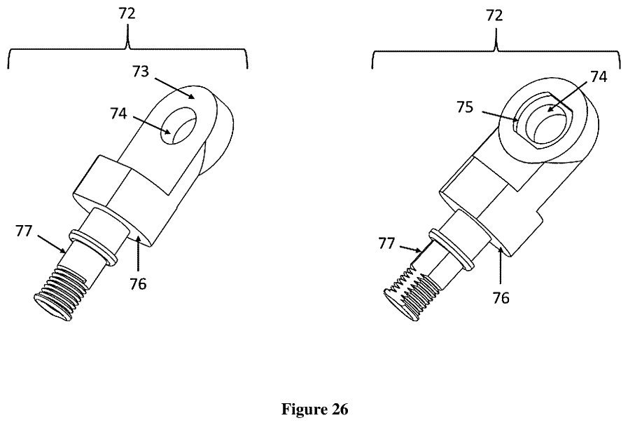

### DETAILED DESCRIPTION

Ring external fixator is constructed by placing two or more rings of either full circumference (1) or half circumference (2) or ⅝^{th} circumference (3) or ⅜^{th} circumference (4) around the limb and connecting them with threaded rods (5). The rings have a flat top surface (6) and a flat bottom surface (7), identical to each other. There is a circular inner surface (8), and a circular outer surface (9), concentric to each other. Ring sizes vary with different inner and outer diameters to accommodate limbs of different sizes. There are multiple round holes (10) in the rings placed at regular intervals connecting the top and bottom surfaces. The holes (10) provide attachment for the threaded connecting rods (5) and other ring fixator components. The top surface (6) and bottom surface (7) of the ring are modified to provide a locking mechanism around each hole (10) in the form of four indentations (11) radiating from the hole.

Rings (1, 2, 3, 4) are made of rigid materials such as metals and alloys or other composites involving carbon fibre, ceramics, and plastic. Threaded rods (5) and various other fixator components described below are made of metals and alloys.

A circular locking washer (12) with a central round hole (13) is used between the ring (1, 2, 3, 4) and fixator components as part of the locking mechanism. On one surface of the locking washer there are four lugs (14) around the central hole (13) that sit in the four indentations (11) on the ring. On the same surface, there are two slots (15) on either side for gripping the locking washer. Other surface of the circular washer has radiating fine serrations (16) around the central hole (13) that interlock with similar radiating serrations on the under surface of the various fixator components described below.

Threaded bone pins (28) inserted in the bone are secured with pin fixation bolt (17). The head (18) of the pin fixation bolt has round cannulation with a diameter matching the diameter of the threaded bone pin (28). The shaft of the pin fixation bolt is fully threaded (19) except at the junction of the head and shaft where it has an unthreaded portion (20). The threaded part of the shaft (19) can be inserted into one of the holes (10) in the ring and secured with a nut. A circular base plate (21) sits around the unthreaded portion (20) of the shaft. The top of the circular base plate has an elevation (22) which sits on either side of the cannulation in the head (18) of pin fixation bolt. The elevation has a curved upper surface (23) with the diameter of the curvature matching the diameter of the threaded bone pin (28). The under surface of the circular base plate has radiating fine serrations (24) that can interlock with similar serrations on the circular washer (16). The elevation (22) of circular base plate has two small holes (25) on either side into which two thin retainer pins (26) are riveted. There is a groove (27) each on either side of the unthreaded portion (20) of the shaft of the pin fixation bolt into which the thin retainer pins (26) project so that the circular base plate (21) is held coupled to the pin fixation bolt (17).

Thus, the interface between the ring (1, 2, 3, 4) and the pin fixation bolt (17) along with an interposed locking washer (12) becomes rotationally stable when the four lugs (14) of the locking washer interlock with the four indentations (11) around the holes in the ring and the serrations on the locking washer (16) interlock with the serrations on the circular base plate (24) of the pin fixation bolt.

The diameter of cannulation in the head (18) of the pin fixation bolt and the diameter of the curved upper surface of the elevation (23) of the circular base plate match the diameter of the threaded bone pin (28). If a smaller diameter threaded bone pin needs to be inserted using a pin fixation bolt with a larger diameter of cannulation in the head (18), adapter half sleeves (29) of appropriate size are used for the purpose. The diameter of the outer surface (30) of the adapter half sleeve matches the diameter of the cannulation in the head (18) of pin fixation bolt. The diameter of the inner surface (31) of the adapter half sleeve matches the outer diameter of the smaller threaded bone pin. An extension (32) at one end of the adapter half sleeve secures it to the head (18) of the pin fixation bolt by gripping it.

Multipurpose posts (33) are modified cubes of various lengths. The base (34) of the multipurpose post has radiating fine serrations on its under surface. A shaft (35) with threads on its outer surface extends downwards from the centre of the base which can be inserted into the holes (10) in the ring with a locking washer (12) interposed between them. The multipurpose post becomes rotationally stable by the interlocking of the serrations on its base (34) with the serrations (16) on the locking washer and by the interlocking of four lugs (14) of the locking washer with the four indentations (11) on the ring. There are one or more round holes (36) in the body of the multipurpose post that provide attachment for pin fixation bolts (17). The two surfaces on either end of the holes (36) are modified to provide a locking mechanism around each hole in the form of four indentations (37) around the hole. When a pin fixation bolt (17) is attached to the multipurpose post (33) with a locking washer (12) interposed between them, it becomes rotationally stable by the interlocking of serrations (24) on the circular base plate with the serrations (16) on the locking washer and by the interlocking of four lugs (14) of the locking washer with the four indentations (37) around the holes on the body of multipurpose post. There is a threaded round hole (38) on the top of the multipurpose post with four radiating indentations around it. Threaded hole (38) can provide attachment to threaded rods (5) or enable insertion of bolts to secure rings (1, 2, 3, 4) and other components. The four indentations around the threaded hole (38) provide a locking mechanism by enabling seating of four lugs (14) of the locking washer.

Plates (39) of various lengths can be used as extensions in the fixator construct. Each plate has a slot (40) and one or more round holes (41) between the top and bottom surfaces of the plate. The top and bottom surfaces of the plate are modified to provide a locking mechanism in the form of multiple indentations (42) radiating around the slot and four indentations (43) around each round hole. The four indentations (43) around each round hole enable seating of four lugs (14) of the locking washer. The multiple radiating indentations (42) around the slot enable seating of four lugs (14) of the locking washer at multiple positions, thus providing a variable position locking mechanism.

Lockable hinge (44) has a first articulating part (45) articulating with second articulating part (46) via a central link shaft (47). The first articulating part has a smooth round hole (48) for the central link shaft (47) around which it can rotate. There are serrations (49) radiating around the smooth round hole (48) on the outer surface of first articulating part that enable reversible locking of the hinge. The inner surface (50) of the first articulating part is smooth to articulate with the second articulating part (46). There is a threaded hole (51) in the base of the first articulating part for insertion of threaded rods (5) or bolts to attach rings (1, 2, 3, 4) and other components. There are serrations (52) radiating around the threaded hole (51) in the base of the first articulating part that enable interlocking with the serrations (16) in the locking washer (12).

The second articulating part has a smooth round hole (53) for the seating of central link shaft (47). There is a recess (54) around the smooth round hole on the outer surface of the second articulating part that locks the central link shaft (47). The inner surface (55) of the second articulating part is smooth to articulate with the first articulating part (45). There is a threaded hole (56) in the base of the second articulating part for insertion of threaded rods (5) or bolts to attach rings (1, 2, 3, 4) and other components. There are serrations (57) radiating around the threaded hole (56) in the base of the second articulating part that enable interlocking with the serrations (16) in the locking washer (12).

Central link shaft (47) is a rod with three parts: A smooth round part (58) at one end, a smooth part with flat surface (59) in the middle and a threaded part (60) at the other end. Smooth round hole of the first articulating part (48) and smooth round hole of the second articulating part (53) articulate around the smooth round part (58) of the central link shaft. Outer end of smooth round part has an expansion (61) that locks into the recess (54) in the outer surface of second articulating part. At the inner end of the smooth round part is the first swaged stopper (62) that stops the first articulating part (45) from coming out. At the outer end of the threaded part (60) of the central link shaft is the second swaged stopper (63). A sliding washer (64) that has a central hole with a flat surface (65), slides on the flat surface of the middle smooth part (59) of the central link shaft, preventing any rotation of the sliding washer (64). There are radiating serrations (66) around the central hole (65) of the sliding washer on its inner surface, that can reversibly interlock with the radiating serrations (49) on the outer surface of the first articulating part. There is a groove (67) around the sliding washer at its outer end. An activator nut (68) sits around the threaded part (60) of the central link shaft and is held in place by the second swaged stopper (63) at the outer end of the threaded part. The activator nut (68) has a groove (69) at its inner end for a retainer C-pin (70). The retainer C-pin (70) passes from the groove (69) in the activator nut into the groove (67) around the sliding washer and couples them together so that the sliding washer (64) slides on the flat surface (59) of the middle smooth part of the central link shaft when the activator nut (68) is turned. When the activator nut (68) is tightened, the sliding washer (64) slides inwards and the serrations (66) on the sliding washer interlock with the serrations (49) on the outer surface of the first articulating part, thus locking the hinge (44). When the activator nut (68) is loosened, the sliding washer (64) slides outwards and unlocks the hinge (44).

Lockable double hinge (71) has two hinge joints placed at right angles to each other. It has two parts identical to the first articulating part (45) of the lockable hinge articulating at either end of a middle articulating part (72). The smooth inner surface (50) of one of the first articulating parts articulates with the smooth inner surface (73) of the middle articulating part. A central link shaft (47) sits in the smooth round hole (74) of the middle articulating part. The expansion (61) on the central link shaft locks into a recess (75) on the outer surface of the middle articulating part. Smooth surface of the base (76) of the middle articulating part articulates with the smooth inner surface (50) of another first articulating part. There is an integrated central link shaft (77) in the centre of the base of the middle articulating part designed identical to the central link shaft (47) of the lockable hinge except that the smooth round part of the integrated central link shaft (77) merges with the base of the middle articulating part. Each of the two hinge joints of the lockable double hinge (71) has a sliding washer (64), an activator nut (68) and a retainer C-pin (70) similar to the lockable hinge (44) providing locking mechanism for the joints. Thus, the two hinge joints of the lockable double hinge (71) articulate with their axes at right angles to each other and can be reversibly locked independent to each other.

### NUMBERED PARTS LISTING

1. Full ring
2. Ring with half circumference
3. Ring with ⅝^{th} circumference
4. Ring with ⅜^{th} circumference
5. Threaded rods
6. Top surface of the ring
7. Bottom surface of the ring
8. Inner surface of the ring
9. Outer surface of the ring
10. Holes in the ring
11. Four indentations around holes in the ring
12. Locking washer
13. Central hole in the locking washer
14. Four lugs around central hole of the locking washer
15. Two slots on either side of the locking washer
16. Serrations radiating from the central hole in the locking washer
17. Pin fixation bolt
18. Cannulated head of the pin fixation bolt
19. Threaded portion of the shaft of pin fixation bolt
20. Unthreaded portion of the shaft of pin fixation bolt
21. Circular base plate
22. Elevation in the circular base plate
23. Curved upper surface on the elevation of circular base plate
24. Radiating serrations on the circular base plate
25. Small holes on the elevation in the circular base plate
26. Thin retainer pin
27. Groove on the unthreaded portion of the shaft of the pin fixation bolt
28. Threaded bone pin
29. Adapter half sleeve for smaller diameter threaded bone pin
30. Outer surface of the adapter half sleeve
31. Inner surface of the adapter half sleeve
32. Gripping extension on the adapter half sleeve
33. Multipurpose Post
34. Base of the multipurpose post with radiating serrations on its under surface
35. Threaded shaft of the multipurpose post
36. Holes in the body of the multipurpose post
37. Four indentations around holes in the body of the multipurpose post
38. Threaded hole on the top of the multipurpose post with four indentations around it
39. Plate
40. Slot in the plate
41. Round holes in the plate
42. Multiple indentations radiating around the slot in the plate
43. Four indentations around each round hole in the plate
44. Lockable hinge
45. First articulating part of lockable hinge
46. Second articulating part of lockable hinge
47. Central link shaft of lockable hinge
48. Smooth round hole in the first articulating part
49. Serrations radiating around the smooth round hole on the outer surface of the first articulating part
50. Smooth inner surface of the first articulating part
51. Threaded hole in the base of the first articulating part
52. Serrations radiating around the threaded hole in the base of the first articulating part
53. Smooth round hole in the second articulating part
54. Recess around the smooth round hole on the outer surface of the second articulating part
55. Smooth inner surface of the second articulating part
56. Threaded hole in the base of the second articulating part
57. Serrations radiating around the threaded hole in the base of the second articulating part
58. Smooth round part of the central link shaft
59. Middle smooth part of the central link shaft with a flat surface
60. Threaded part of the central link shaft
61. Expansion at the outer end of the smooth round part of the central link shaft
62. First swaged stopper at the inner end of the smooth round part of the central link shaft
63. Second swaged stopper at the outer end of the threaded part of the central link shaft
64. Sliding washer of the lockable hinge
65. Central hole with flat surface in the sliding washer
66. Serrations radiating around the central hole of the sliding washer on its inner surface
67. Groove around the sliding washer at its outer end
68. Activator nut of the lockable hinge
69. Groove in the activator nut at its inner end
70. Retainer C-pin of the lockable hinge
71. Lockable double hinge
72. Middle articulating part of lockable double hinge
73. Smooth inner surface of the middle articulating part
74. Smooth round hole in the middle articulating part
75. Recess around the smooth round hole on the outer surface of the middle articulating part
76. Smooth surface of the base of the middle articulating part
77. Integrated central link shaft of the middle articulating part

1-48. (canceled)

49. A reversible locking mechanism for a ring external fixator that is configured to attach a threaded bone pin to a hole in the ring external fixator, wherein the reversible locking mechanism is located at an interface between the ring external fixator and ring external fixator components so that two opposing surfaces interlock with each other to provide rotational stability for the ring external fixator components, and wherein the ring external fixator components are selected from a group consisting of pin fixation bolts, multipurpose bolts, plates, and hinges.

50. The reversible locking mechanism for a ring external fixator of claim 49, wherein the reversible locking mechanism is configured to attach a threaded bone pin to a hole in the ring external fixator comprising four indentations, wherein the reversible locking mechanism comprises a pin fixation bolt assembly and a circular locking washer;
wherein the pin fixation bolt assembly comprises:
a head having a round cannulation with a diameter that matches a diameter of the threaded bone pin;
a shaft that is fully threaded except at a junction of the head and the shaft; and
a circular base plate sat around the unthreaded portion of the shaft, the circular base plate having an elevation with a curved upper surface that matches the diameter of the threaded bone pin and which sits on either side of the cannulation, and an under surface with radiating fine serrations;
wherein the circular locking washer comprises:
a first surface on which four lugs and two slots are arranged around a central hole of the washer; and
a second surface on which radiating fine serrations are arranged around the central hole,
wherein, the combined effect of the four lugs on the circular locking washer interlocking with the four indentations around the hole in said ring external fixator ring and the serrations of the circular base plate and the circular locking washer interlocking together provides a rotationally stable connection between the threaded bone pin and the ring external fixator ring.

51. The reversible locking mechanism for a ring external fixator of claim 50, further comprising two thin retainer pins riveted into two small holes on either side of the elevation of circular base plate and project into a groove each on either side of the unthreaded portion of the shaft of the pin fixation bolt, such that the circular base plate is held coupled to the pin fixation bolt.

52. The reversible locking mechanism for a ring external fixator of claim 50, wherein a smaller diameter threaded bone pin can be held by a pin fixation bolt with a larger diameter of cannulation in the head via adapter half sleeves where, an extension at one end of the adapter half sleeve secures the adapter half sleeves to the head of the pin fixation bolt by gripping it.

53. The reversible locking mechanism for a ring external fixator of claim 52, wherein a diameter of an outer surface of the adapter half sleeves matches a diameter of the cannulation in the head of pin fixation bolt, and a diameter of an inner surface of the adapter half sleeve matches an outer diameter of the smaller threaded bone pin.

54. The reversible locking mechanism for a ring external fixator of 49, wherein the reversible locking mechanism is configured to attach a threaded bone pin to a hole in a ring external fixator that comprises four indentations, wherein the reversible locking mechanism comprises a multipurpose post and a circular locking washer;
wherein the multipurpose post comprises a modified cube with a base that has fine radiating serrations on its under surface around a shaft with threads on its outer surface extending downwards from a center of the base which can be inserted into the holes in the ring external fixator with a locking washer interposed between them; and
wherein the multipurpose post is rotationally stabilized via the interlocking of the serrations on its base with the serrations on the locking washer and by the interlocking of four lugs of the locking washer with the four indentations on the ring.

55. The reversible locking mechanism for a ring external fixator of claim 54, wherein there is at least one hole in the multipurpose post configured for attachment of the pin fixation bolts, and two surfaces on either end of the at least one hole are configured to provide a locking mechanism around each hole in the form of four indentations around the hole.

56. The reversible locking mechanism for a ring external fixator of claim 54, wherein, when a pin fixation bolt is attached to the multipurpose post with a locking washer interposed between them, said pin fixation bolt is rotationally stabilized by the interlocking of serrations on the circular base plate with the serrations on the locking washer and by the interlocking of four lugs of the locking washer with the four indentations around the holes on the body of multipurpose post.

57. The reversible locking mechanism for a ring external fixator of claim 54, wherein a threaded round hole on the top of the multipurpose post if configured to provide attachment to threaded rods or to enable insertion of bolts to secure rings and other components and the four indentations around the threaded hole provide a locking mechanism by enabling seating of four lugs of the locking washer.

58. The reversible locking mechanism for a ring external fixator of claim 49,
wherein the reversible locking mechanism is configured to attach a threaded bone pin to a hole in the ring external fixator that comprises four indentations,
wherein the locking mechanism comprises a plate that can be used as an extension to the ring external fixator and a circular locking washer;
wherein the plate includes a slot and one or more round holes between a top surface and a bottom surface of the plate,
wherein the top and bottom surfaces of the plate are configured to provide a locking mechanism in the form of six to twelve indentations radiating around the slot and four indentations radiating around each round hole; and
wherein the four indentations around each round hole in the plate enable seating of four lugs of the locking washer and six to twelve radiating indentations around the slot in the plate enable seating of four lugs of the locking washer at multiple positions, thereby providing a variable position locking mechanism.

59. The reversible locking mechanism for a ring external fixator of claim 49, wherein the reversible locking mechanism is configured to attach a threaded bone pin to a hole in a ring external fixator that comprises four indentations,
wherein the reversible locking mechanism comprises a lockable hinge and a circular locking washer;
wherein the lockable hinge comprises a first articulating part articulating with a second articulating part via a central link shaft that incorporates a lock comprising a sliding washer and an activator nut;
wherein the first articulating part comprises a first smooth round hole and the second articulating part comprises an second smooth round hole for the seating of central link shaft around which the first and second articulating parts can rotate;
wherein an inner surface of the first articulating part is smooth so as to articulate with the second articulating part and an inner surface of the second articulating part is smooth so as to articulate with the first articulating part;
wherein there are fine radiating serrations around the smooth round hole on an outer surface of first articulating part that enable reversible locking of the hinge and there is a recess around the smooth round hole on an outer surface of the second articulating part that locks the central link shaft;
wherein the central link shaft comprises a rod with three parts: a smooth round part at one end, a smooth part with flat surface in the middle and a threaded part at the opposite end;
wherein the smooth round hole in the first articulating part and the smooth round hole in the second articulating part each articulate around the smooth round part of the central link shaft;
wherein the outer end of smooth round part of the central link shaft comprises an expansion to lock into the recess in the outer surface of second articulating part;
wherein at the inner end of the smooth round part of the central link shaft is arranged a first swaged stopper that stops the first articulating part from coming out;
wherein a sliding washer that has a central hole with a flat surface, is arranged to slide on the flat surface of the middle smooth part of the central link shaft to prevent rotation of the sliding washer,
wherein the fine radiating serrations around the central hole of the sliding washer on its inner surface reversibly interlock with the serrations on the outer surface of the first articulating part, to stop its movement;
wherein an activator nut sits around the threaded part of the central link shaft, and is held in place by a second swaged stopper at the outer end of the threaded part of the central link shaft;
wherein a retainer C-pin is arranged to pass from a groove in the activator nut at its inner end into a groove around the sliding washer at its outer end, thereby coupling them together so that the sliding washer slides on the flat surface of the middle smooth part of the central link shaft when the activator nut is turned;
wherein when the activator nut is tightened, the sliding washer slides inwards and the serrations on the sliding washer interlock with the serrations on the outer surface of the first articulating part, thus locking the hinge and when the activator nut is loosened, the sliding washer slides outwards and unlocks the hinge;
wherein a threaded hole is defined in a base of the first articulating part and a threaded hole in a base of the second articulating part for insertion of threaded rods or bolts to attach rings and other components; and
wherein there are fine radiating serrations around the threaded hole in the base of the first articulating part and fine radiating serrations around the threaded hole in the base of the second articulating part to enable interlocking with the serrations in the circular locking washer.

60. A ring external fixator with reversible locking mechanism, the ring external fixator comprising:
a plurality of rings that are connected together by a plurality of threaded rods, wherein each ring comprises at least one threaded bone pin attached thereto using the reversible locking mechanism of claim 49;
wherein each ring further comprises:
a flat top surface and a flat bottom surface, identical to each other;
a circular inner surface and a circular outer surface, concentric to each other; and
a plurality of round holes arranged at regular intervals, connecting top and bottom surfaces of each ring,
wherein the top surface and bottom surface of each ring define four indentations radiating from each hole; and
wherein an interface between each ring, an interposed locking washer, and at least one ring external fixator component selected from the group consisting of pin fixation bolts, multipurpose bolts, plates, and hinges, becomes rotationally stable when the four lugs of the locking washer interlock with the four indentations around the hole in the ring and the serrations on the locking washer interlock with the serrations on each of said ring external fixator components.

61. The ring external fixator of claim 60, wherein the ring external fixator is constructed by placing two or more full circumference or half circumference or ⅝^{th} circumference or ⅜^{th} circumference rings around a limb of a patient and connecting them with the threaded rods.

62. The ring external fixator of claim 60, wherein the ring sizes vary with different inner and outer diameters to accommodate limbs of different sizes.
